(19) United States
(12) Patent Application Publication
(10) Pub. No.: US 2024/0145030 A1
(43) Pub. Date: May 2, 2024

(54) SYSTEMS AND METHODS TO PREDICT PROTEIN-PROTEIN INTERACTION

(71) Applicant: Triana Biomedicines, Inc., Lexington, MA (US)

(72) Inventor: Johannes Maier, Montreal (CA)

(21) Appl. No.: 18/376,729

(22) Filed: Oct. 4, 2023

Related U.S. Application Data
(60) Provisional application No. 63/414,233, filed on Oct. 7, 2022.

(51) Int. Cl. G16B 15/30; G06N 3/02

(52) U.S. Cl. CPC G16B 15/30; G06N 3/02

(57) ABSTRACT

Systems and methods to predict protein-protein interaction are provided herein for predicting one or more hot spots on a surface of a protein. An example method can include receiving a 3D model representing a whole structure of a protein. The method can include determining different surface patches associated with the 3D model. The method can include determining for at least one of different surface patches at least one of a geometric property or a chemical property. The method can further include assigning each node of a surface patch input features including chemical features. The method can include processing, with a neural network, at least one of a collection of geometric properties collected from one or more of different surface patches or a collection of the chemical properties collected from one or more of different the surface patches to predict one or more hot spots on the surface of the protein.

### CROSS REFERENCE TO RELATED APPLICATIONS

The present application claims the benefit of U.S. Provisional Patent Application No. 63/414,233, filed on Oct. 7, 2022, which is incorporated herein by reference in its entirety.

### BACKGROUND

Protein-protein interactions (PPIs) underlie most biological processes and have pivotal roles in normal functions of the proteins in all organisms. Predicting these interactions is crucial for understanding most biological processes, such as DNA replication and transcription, protein synthesis and secretion, signal transduction and metabolism, and in the development of new drugs. Since proteins are large molecules with complex three-dimensional structures, PPIs are highly specific in that they require a multitude of suitable interactions between each partner, e.g., proper hydrogen bonding, electrostatic interactions, and hydrophobicity. Thus, predicting PPIs using a computational method can be challenging and resource intensive.

### SUMMARY

The present disclosure relates to improved systems and methods to predict protein-protein interaction by predicting one or more hot spots as defined below.

The systems and methods taught herein address some of the technical problems of conventional protein-protein interaction prediction using computational methods in conjunction with training of a neural network. The conventional systems and methods for predicting PPIs have several drawbacks. For example, a conventional neural network, when executing a machine-learning based algorithm, learns from a generic protein benchmark data set but has no specific awareness of small molecule binding sites or weak protein-protein binding. Furthermore, it is often difficult to introduce fundamental changes regarding input features and retrain the neural network at least due to simple and unclear definitions for parameters used for calculating geometric and chemical properties. For example, identifying a correct binding pose of a protein complex and systematically distinguishing it from an extremely large pool of plausible binding configurations is widely accepted as a hugely complex challenge. Conventionally, algorithms and methods based on physics principles are computationally feasible using scoring functions with various levels of abstractions that in turn often lead to incorrect predictions of the binding pose. To address the problems of the conventional systems and methods, embodiments of the present disclosure improve accuracy of calculating geometric and chemical properties of the entire three-dimensional (3D) structures representing whole proteins. Improvements to the 3D structures representing whole proteins are accomplished by providing clear definitions for parameters used for calculating geometric and chemical properties, more accurate parameters (e.g., atom partial charges and radii, atomic s log P propensity values, etc.) for calculating geometric and chemical properties, and more accurate molecular surface representations. Additional improvements to predicting PPIs as taught herein include allowing flexibility in modifying chemistry features informative of protein binding, normalization of input features that allows assignment user-defined feature weights used for optimizing the neural network. As discussed in more detail below, a training data set for training a network to predict PPI is improved. For example, a training data set as taught herein treats a whole protein as a 3D model. Further, a training data set as taught herein allows the model to take into account in predicting PPI weak binding scenarios. Still further, a training data set as taught herein moves away from the conventional practice of relying on (and in some embodiments eliminating) a residue constraint when predicting PPI weak binding scenarios. The ability of a model taught herein to minimize or eliminate residue constraints provides the ability to add small molecule ligand related data to the training set.

In one embodiment, the present disclosure provides an example method to predict one or more hot spots on a surface of the protein that are highly likely involved in PPI. The method includes receiving a 3D model representing a whole structure of a protein. The method includes determining a plurality of different surface patches associated with the 3D model. A surface patch as used herein is defined below. The method includes determining for each of the plurality of different surface patches at least one of a geometric property or a chemical property. The method further includes processing, with a neural network, at least one of a collection of geometric properties collected from one or more of the plurality of different surface patches or a collection of the chemical properties collected from one or more of the plurality of different the surface patches to predict one or more hot spots on a surface of the protein that is highly likely to be involved in an interaction between the protein and a biomolecule. A biomolecule as used herein is defined below.

In another embodiment, the present disclosure provides an example method for training a neural network for a protein-biomolecule interaction prediction. The method includes training a neural network using a training set having a corpus of 3D models. Each 3D model represents a whole structure of an identified protein and having a plurality of different surface patches. Each of the plurality of different surface patches includes at least one of a geometric property or a chemical property associated with the identified protein. The method further includes deploying the trained neural network to predict one or more hot spots on a surface of a protein that is highly likely involved in an interaction between the protein and a biomolecule (e.g., a protein, a RNA, DNA, or the like).

### BRIEF DESCRIPTION OF THE DRAWINGS

The foregoing features of the present disclosure will be apparent from the following Detailed description of the present disclosure, taken in connection with the accompanying drawings, in which:

FIG. 1 is a diagram illustrating an example embodiment of the system of the present disclosure;

FIG. 2A is a flowchart illustrating overall processing steps carried out by the system of the present disclosure;

FIG. 2B is a flowchart illustrating example processing steps carried out by the system of the present disclosure for predicting a hot spot;

FIG. 3A illustrates example surface patches in a molecular surface representation of a protein;

FIG. 3B is a zoomed in illustration of one of the surface patches in FIG. 3A;

FIG. 4A illustrates an example molecular surface representation representing positive charges of the protein in FIG. 3A;

FIG. 4B illustrates an example molecular surface representation representing negative charges of the protein in FIG. 3A;

FIG. 5 illustrates an example molecular surface representation representing hydrophobicity properties of the protein in FIG. 3A;

FIG. 6A illustrates an example molecular surface representation representing hydrogen-bond acceptor regions of the protein in FIG. 3A;

FIG. 6B illustrates an example molecular surface representation representing hydrogen-bond donor regions of the protein in FIG. 3A;

FIG. 7 illustrates a hydrogen bond geometry used in a hydrogen-bond energy potential given in Equation (1);

FIG. 8 illustrates an example molecular surface representation of a protein interface surface;

FIG. 9 is an example flowchart illustrating neural network training steps carried out by the system of the present disclosure;

FIGS. 10A-10D are Table 1 showing an example training set of protein-protein interaction pairs;

FIGS. 11A-11C are Table 2 showing an example training set of protein-DNA/RNA interaction pairs;

FIG. 12 is an example diagram illustrating computer hardware and network components on which the system can be implemented; and

FIG. 13 is an example block diagram of an example computing device that can be used to perform one or more steps of the methods provided herein.

### DETAILED DESCRIPTION

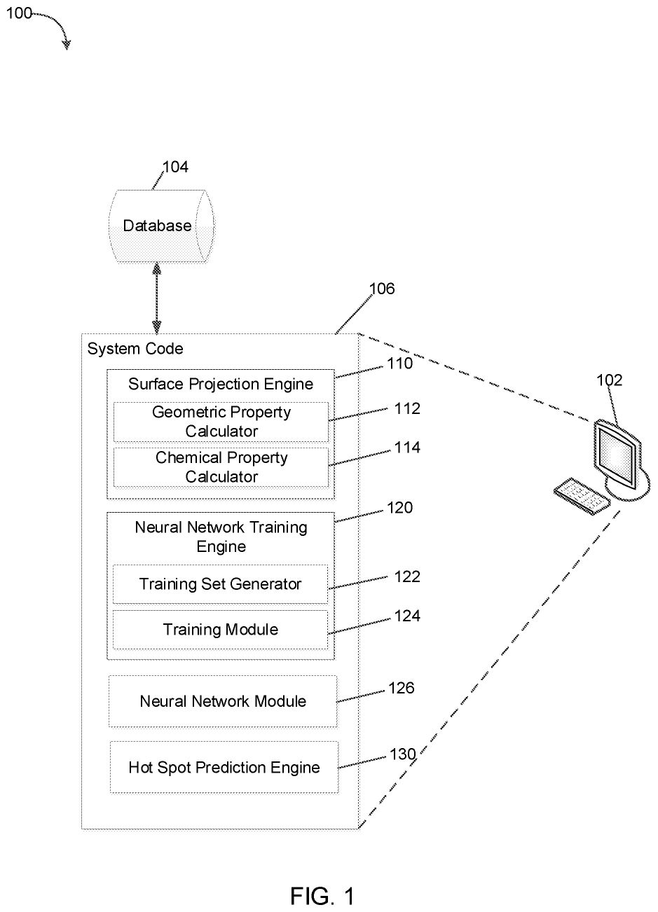

The present disclosure relates to systems and methods for predicting protein-protein interaction. Example systems and methods are described in detail below in connection with FIGS. 1-13.

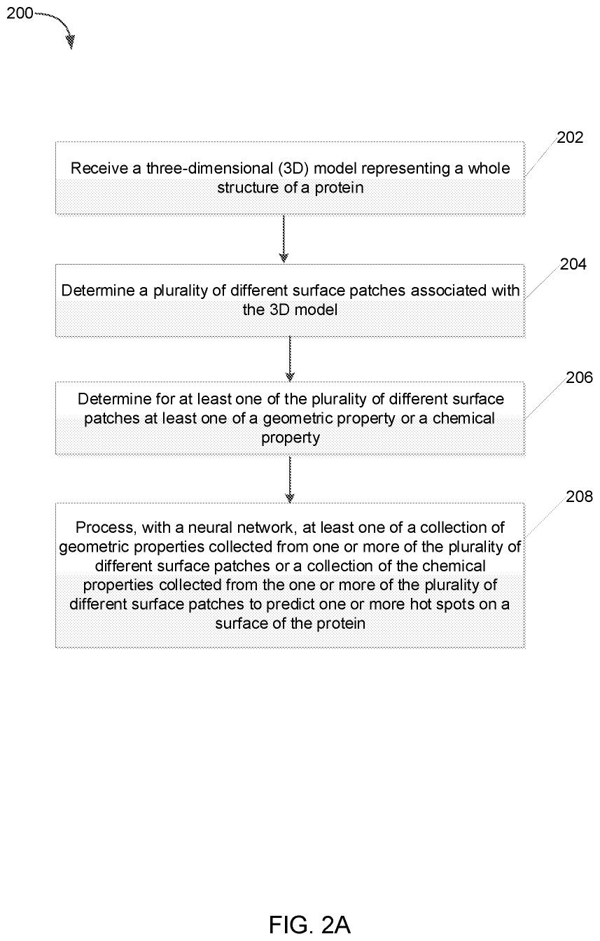

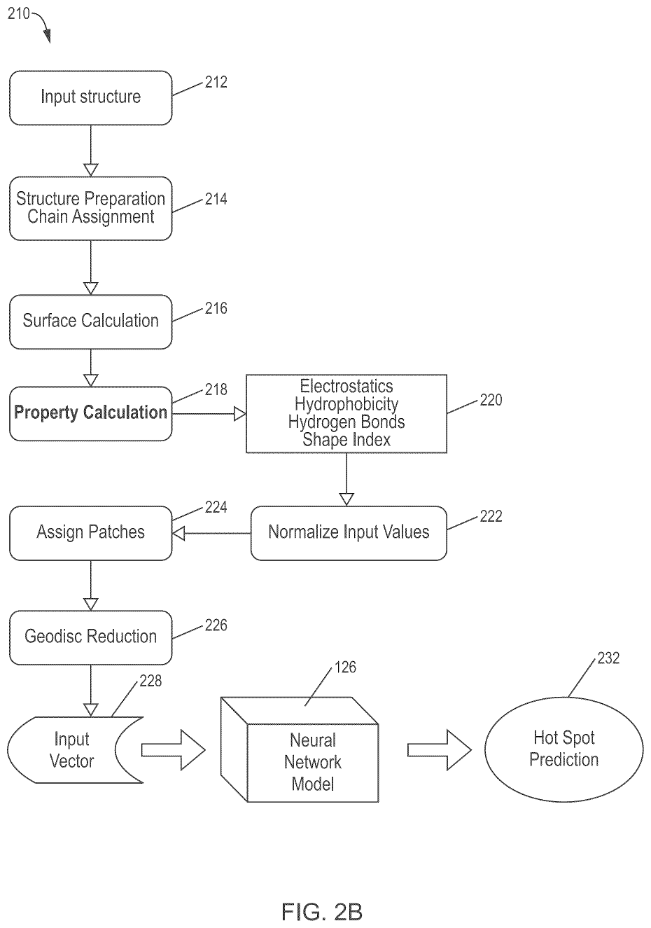

PPIs are physical contacts of high specificity established between two or more protein molecules as a result of binding events steered by interactions that include but not limited to electrostatic forces, hydrogen bonding and the hydrophobic effect. Predicting interactions between proteins and other biomolecules solely based on structure remains a challenge in biology. The systems and methods of the present disclosure utilize a neural network trained on geometric and/or chemical property data projected onto an interaction surface of a protein to predict one or more hot spots on a surface of the protein. The systems and methods provide several advantages compared with the conventional methods including, but not limited to optimized surface projections using improved geometric and/or chemical property data in electrostatics, hydrophobicity, and hydrogen bonds. Disclosed herein, are improved training set(s) that improve the accuracy of the models representing the protein structure. In some embodiments, the improved protein models have weak protein interactions scenarios (e.g., interactions having dissociation constant K_{D} greater than 10⁻⁴ M indicative of weak binding and/or low affinity), improved surface patch definitions instead of arbitrary radii as further described below with respect to FIGS. 2A and 2B, or the like. In some embodiments, the improved protein models have weak or strong interactions between a protein and a biomolecule.

As used herein, “protein—protein interactions” (PPIs) are specific, physical, and intentional interactions between the interfaces of two or more proteins as the result of biomolecular events/forces. The interaction interface should be non-generic, i.e., evolved for a purpose distinct from generic functions such a protein production, degradation, aggregate formation, and the like. In one aspect, the biomolecular events/forces include one or more covalent or non-covalent interactions such as, e.g., hydrogen bonding, electrostatic interactions, hydrophobic interactions, etc.

As used herein, a “hot spot” refers to a specific region on a protein surface, the specific region being more likely than not to result in a useful protein to protein interaction. More specifically, a “hot spot” refers to a collection of residues that makes a significant contribution to the binding free energy of a protein.

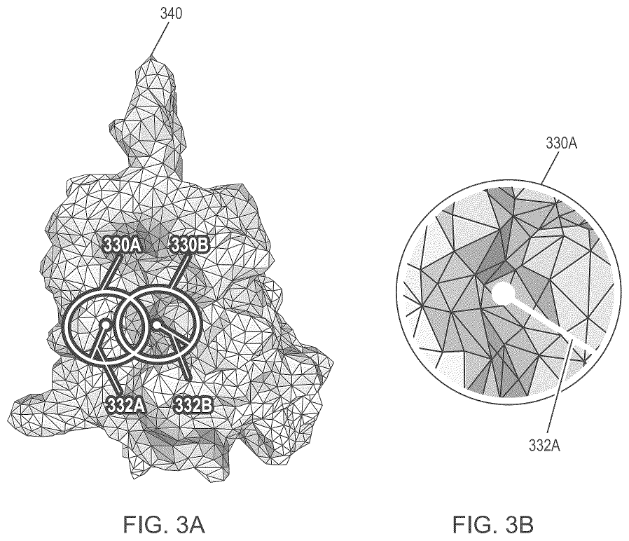

As used herein, a “surface patch” is defined by a collection of mesh elements (e.g., a polygon mesh element having vertices, edges, faces, etc.) pooled as a result of application of district criterion (e.g., a collection of surface points with similar and/or predefined geometric/chemical properties). An example of a “surface patch” is discussed below in relation to FIGS. 3A and 3B.

As used herein, an “interaction patch” is defined as a collection of surface points of a coherent region of a particular type (e.g., positive charge, negative charge, hydrophobicity) involved in protein-protein interactions.

As used herein, a “biomolecule” refers to a molecule which is produced by a living organism and includes, but is not limited to carbohydrates, proteins, nucleic acids (DNA and RNA), lipids and polysaccharides.

As used herein, a “molecular surface” refers to a surface which an exterior probe-sphere touches as it is rolled over the spherical atoms of that molecule.

As used herein, a “neural network” refers to an artificial neural network having an input layer, one or more hidden layers, and an output layer. Each node, or artificial neuron, connects to another and has an associated weight and threshold. If the output of any individual node is above the specified threshold value, that node is activated, sending data to the next layer of the network. Otherwise, no data is passed along to the next layer of the network. A convolutional neural network (CNN) is a class of a neural network in which the hidden layers include convolution layers that convolve an input and pass its result to a next layer, pooling layers that reduce dimensions of data by combining outputs of neuron clusters at one layer into a single neuron in a next layer, fully connected layers that connect every neuron in one layer to every neuron in another layer.

Turning to the drawings, FIG. 1 is a diagram illustrating an example embodiment of the system 100 of the present disclosure. The system 100 can be embodied as a central processing unit 102 (processor) in communication with a database 104. The processor 102 can include, but is not limited to, a computer system, a server, a personal computer, a cloud computing device, a smart phone, or any other suitable device programmed to carry out the processes disclosed herein. Still further, the system 100 can be embodied as a customized hardware component such as a field-programmable gate array (“FPGA”), an application-specific integrated circuit (“ASIC”), embedded system, or other customized hardware components without departing from the spirit or scope of the present disclosure. It should be understood that FIG. 1 is just one potential configuration, and that the system 100 of the present disclosure can be implemented using a number of different configurations.

As taught herein, when predicting protein-protein interaction the whole protein is modeled as a 3D model. The database 104 includes various types of data including, but not limited to, three-dimensional (3D) models, each 3D model representing a whole structure of a protein, preprocessed geometric property data and/or chemical property data associated with one or more 3D models, trained neural network(s), and one or more outputs from various components of the system 100 (e.g., outputs from a surface projection engine 110, a geometric property calculator 112, a chemical property calculator 114, a neural network training engine 120, a training set generator 122, a training module 124, a neural network module 126, a hot spot prediction engine 130, and/or other suitable components of the system 100).

A protein structure is built as a plurality of chains of amino acids that are folded into a unique 3D shape. The plurality of chains of amino acids can be divided into side chains and a main chain (also referred to as a protein backbone). The amino acids are small organic molecules that consist of an alpha (central) carbon atom linked to an amino group, a carboxyl group, a hydrogen atom, and a variable component. An alpha carbon atom linked to a variable component forms a side chain. Within a protein, multiple amino acids are linked together by peptide bonds, thereby forming a long chain. Once linked in the protein, an individual amino acid is called a residue, the linked series of carbon, nitrogen, and oxygen atoms are known as the main chain or protein backbone, and the linked series of carbon atoms and variable components are known collectively as side chains. Multiple side chains have a great variety of chemical structures and properties. It is the combined effect of all of the amino acid side chains in a protein that ultimately determines its three-dimensional structure, its chemical reactivity and propensity to engage a protein-biomolecule interaction.

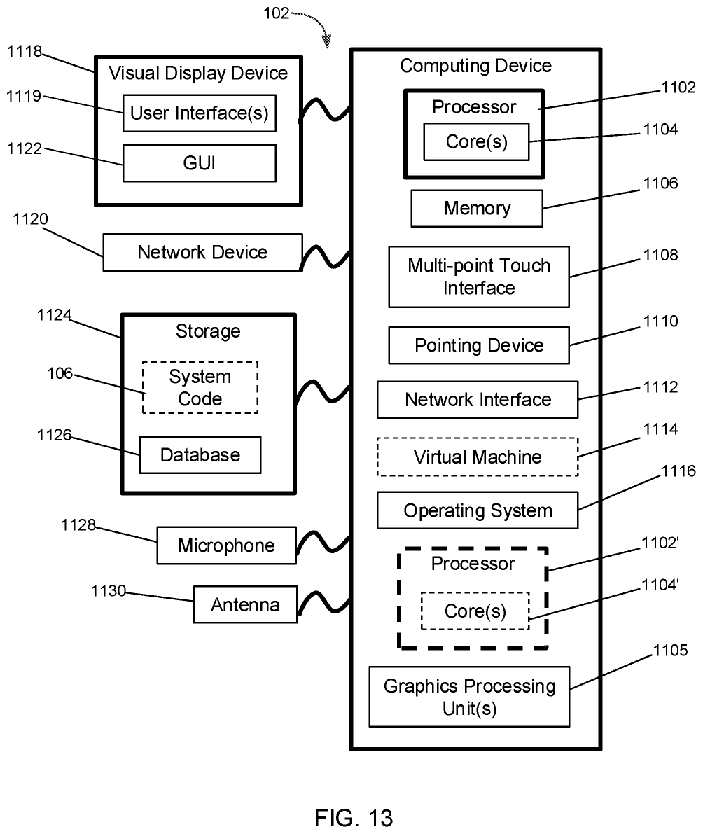

The system 100 includes system code 106 (non-transitory, computer-readable instructions) stored on a computer-readable medium, for example storage 1124 in FIG. 13, and executable by the hardware processor 102 or one or more computer systems. The system code 106 can include various custom-written software modules that carry out the steps/processes described herein, and can include, but is not limited to, the surface projection engine 110, the geometric property calculator 112, the chemical property calculator 114, the neural network training engine 120, the training set generator 122, the training module 124, a neural network module 126, and the hot spot prediction engine 130. Each component of the system 100 is described with respect to FIGS. 2-13.

The system code 106 can be programmed using any suitable programming languages including, but not limited to, C, C++, C#, Java, Python, or any other suitable language. Additionally, the system code 106 can be distributed across multiple computer systems in communication with each other over a communications network, and/or stored and executed on a cloud computing platform and remotely accessed by a computer system in communication with the cloud platform. The system code 106 can communicate with the database 104, which can be stored on the same computer system as the system code 106, or on one or more other computer systems in communication with the system code 106.

FIG. 2A is a flowchart illustrating overall processing steps 200 carried out by the system 100 of the present disclosure. In step 202, the system 100 receives a 3D model representing a whole structure of a protein (e.g., the protein structure described above with respect to FIG. 1). For example, the system 100 can retrieve a 3D model from the database 104.

In step 204, the system 100 determines a plurality of different surface patches associated with the 3D model. For example, the surface projection engine 110 of the system 100 can compute a molecular surface (e.g., solvent-excluded surface, solvent-accessible surface, discretized molecular surface, or the like) from the 3D model, and generate a molecular surface representation to visualize the molecular surface. A molecular surface is defined above. The generated molecular surface representation (e.g., a polygon mesh having a plurality of mesh elements) can include a plurality of different surface patches. For example, a surface patch can be the result of collecting surface points based on a predefined geodesic radius (e.g., 5 angstroms (Å), 9 Å, 12 Å or other suitable geodesic radius greater than 12 Å). A geodesic radius is a distance from a center of a geodesic circle on a surface to points on the geodesic circle. The system 100 can determine a geodesic radius based on specific applications or specific interaction types. For example, in some applications (e.g., a PPI search, pocket classification), the system 100 can select 12 Å as a geodesic radius to cover a surface area of many PPIs. In some embodiments, the system 100 can select 5 Å or 9 Å as a geodesic radius to generate small surface patches. In some embodiments, instead of selecting a surface patch with a predefined geodesic radius, the system 100 determines an interaction patch as a collection of coherent regions of a distinct biophysical type (e.g., positive and negative charge, hydrophobicity) involved in protein-protein interactions. The system 100 can also factor in just vertices of surface patches having the same biophysical type at a predefined distance radius (e.g., 5 Å or 9 Å) into a neural network.

In some embodiments, the system 100 can input information from the same surface patch into a neural network for processing. Examples of a molecular surface representation and surface patches are shown in FIGS. 3A and 3B.

In step 206, the system 100 determines for at least one of the plurality of different surface patches at least one of a geometric property or a chemical property. In some embodiments, the system 100 determines for each of the plurality of different surface patches at least one of a geometric property or a chemical property. For example, for each point of a surface patch (e.g., a vertex of each mesh element in the surface patch), the geometric property calculator 112 of the system 100 can calculate geometric properties, and the chemical property calculator 114 of the system 100 can compute chemical properties. Examples of a geometric property can include a shape index (describes a shape around each point on the surface patch with respect to the local curvature), and a distance-dependent curvature (describes a relationship between a distance to a center of a surface patch and surface normals of each point and the center point).

Examples of a chemical property can include properties associated with electrostatics, hydrophobicity, and hydrogen bonds.

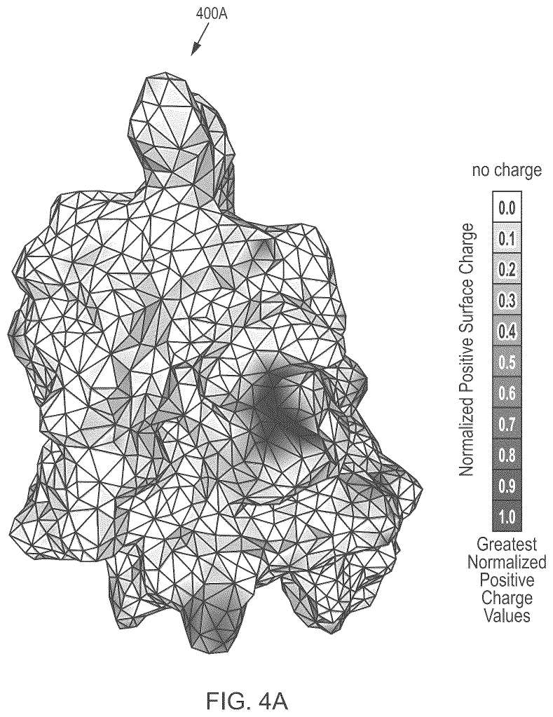

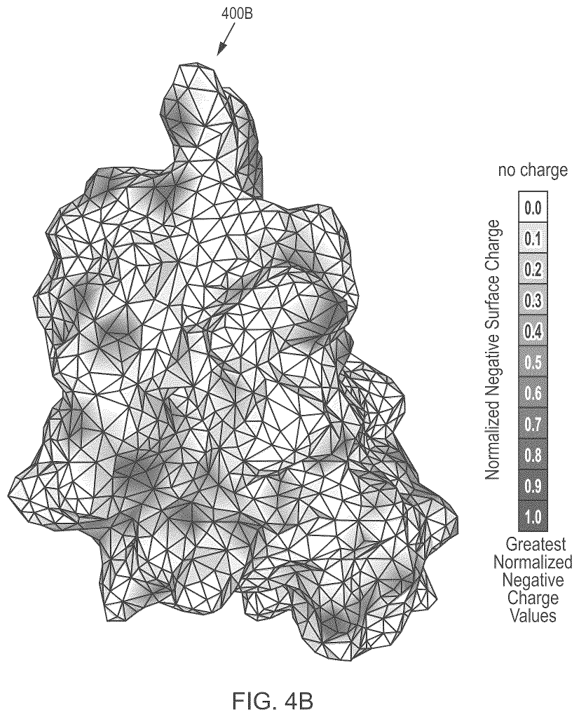

The properties associated with electrostatics can include atom partial charges and radii assigned by a protein force field (e.g., Amber99 force fields or other suitable AMBER (assisted model building and energy refinement) force fields), rather than small molecular force field that is used in conventional systems and methods. Use of the protein force field by the methods and systems taught herein can improve the accuracy of properties associated with electrostatics. The system 100 can set pH values and remove clashes prior to electrostatics calculation, which can reduce errors caused by a coarse surface grid used in the conventional systems. An example of a molecular representation having electrostatics properties is described with respect FIGS. 4A and 4B.

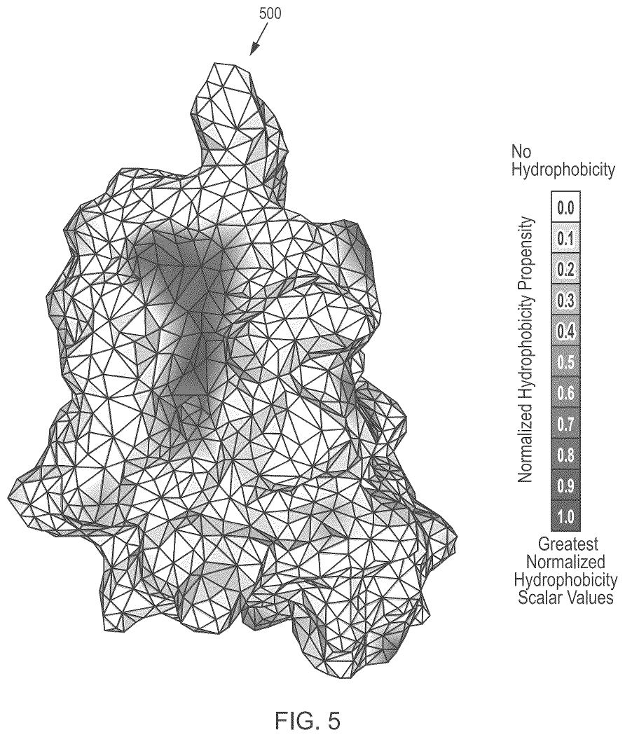

The properties associated with hydrophobicity can include atomic s log P (enhance atomic or hybrid partition confident for n-octanol/water) propensity values that are atom-based and independent of natural amino acid context, and allow for reliable predictions with small molecule present instead of using Kyte-Doolittle residue propensities for hydrophobicity used in conventional methods that is simplistic and depends on a reduce context. An example of a molecular representation having hydrophobicity properties is described with respect to FIG. 5.

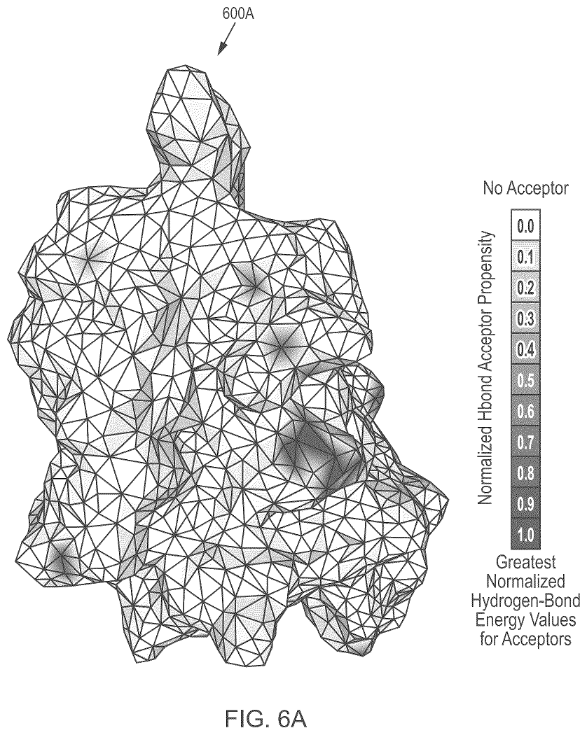

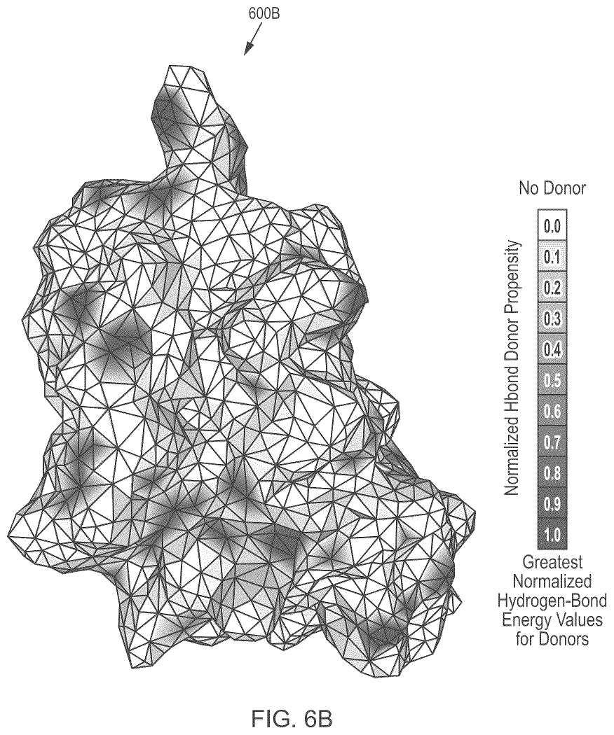

As taught herein, the properties associated with hydrogen bonds can include a negative value representing a donor, a positive value representing an acceptor, a hydrogen bond geometry defined by an established force field definition, and a hydrogen bond energy defined by the established force field definition as described below. By comparison, conventional methods which are prone to errors caused by subtle changes on surface atoms often use obscure definitions for the hydrogen bond geometry and energy scale. An example of a molecular surface representation having hydrogen bonds properties is described with respect to FIGS. 6A and 6B.

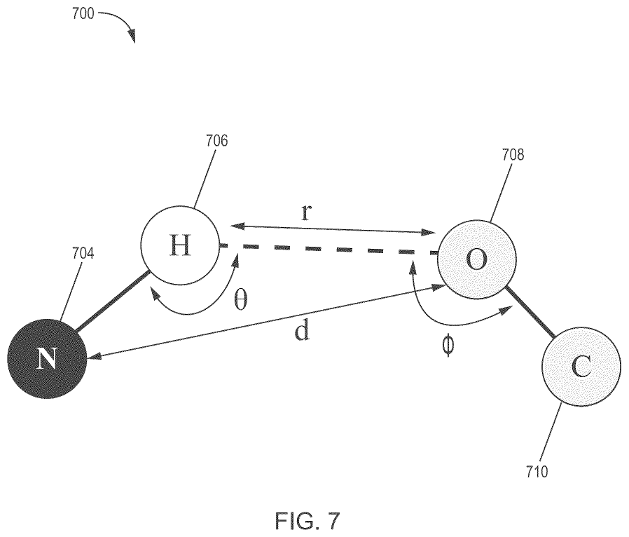

In some embodiments, a hydrogen bond energy calculation is based on the Equation (1) as follows:

$${E}_{HB} = {V}_{0} \{ 5 {( \frac{{d}_{0}}{d} )}^{12} - 6 {( \frac{{d}_{0}}{d} )}^{10} \} F ( θ , ϕ , γ ) \qquad ( 1 )$$

Where, for a pair of sp³ donor and sp³ acceptor, F=cos² θ exp(−[π−θ]⁶) cos² (ϕ−109.5); for a pair of sp³ donor and sp² acceptor, F=cos² θ exp(−[π−θ]⁶) cos² (ϕ); for a pair of sp² donor and sp³ acceptor, F={cos² exp(−[π−θ]⁶)}²; for a pair of sp² donor and sp² acceptor, F=cos² θ exp(−[π−θ]⁶) cos² (max [ϕ, γ]), and V₀=8 kilocalories per mole (kcal/mol), and d₀=2.8 Å. An example of relationships among do, 9, 0, donor, acceptor and hydrogen is described with respect to FIG. 7.

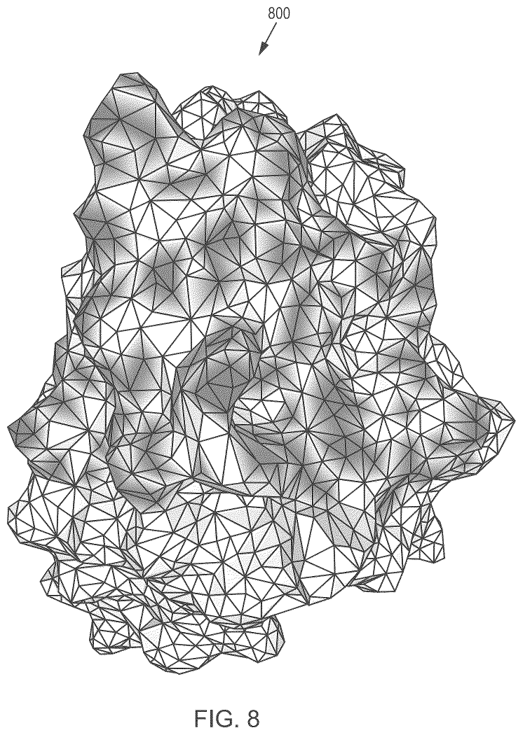

In some embodiments, the system 100 also refines interface input definitions. The system 100 considers surface vertices of atoms that are in contact with other chains (e.g., other side chains or the main chain) of the protein. Consequently, the computational efficiency is improved, at least because the atoms that are not in contact with other chains are not computed. An example molecular surface representation 800 representing atoms in contact with other chains of a protein is illustrated with respect to FIG. 8.

In step 208, the system 100 processes, with a neural network 126, at least one of a collection of geometric properties collected from one or more of the plurality of different surface patches or a collection of the chemical properties collected from the one or more of the plurality of different surface patches or both are used to predict one or more hot spots on a surface of the protein that are highly likely involved in an interaction between the protein and a biomolecule. The hot spot prediction engine 130 of the system 100 can input the 3D surface patches having geometric properties and/or chemical properties to the neural network (e.g., convolutional neural network, geometric deep learning, or other similar algorithms) though an input layer, hidden layers (e.g., convolutional layers followed by a series of fully connected layers) and an output layer. The neural network converts the 3D surface patches into feature descriptors (e.g., a number, a vector, a matrix or a string), and further process the feature descriptors to predict one or more hot spots.

In some embodiments, the hot spot prediction engine 130 can compare an output of the neural network with a hot sport threshold to determine whether or not an input surface patch is highly likely to be a hot spot. A hot spot threshold refers to a value or a value range indicating that an input surface patch is highly likely to be a hot spot. For example, if the hot spot prediction engine 130 determines that an output of the neural network satisfies the hot spot threshold, the hot spot prediction engine 130 determines that an input surface patch is highly likely to be a hot spot. If the hot spot prediction engine 130 determines that an output of the neural network dissatisfies the hot spot threshold, the hot spot prediction engine 130 determines that an input surface patch is not likely to be a hot spot.

In some embodiments, the neural network can predict one or more hot spots of a particular type (e.g., positive charge, negative charge, or hydrophobicity). For example, the neural network can place the predicted hot spots into a classification indicative of a particular type.

In some embodiments, the process of converting the 3D surface patches into 2D feature descriptors (also referred to as a dimensionality reduction) can be performed by several neural network algorithms including, but not limited to, multidimensional scaling (MDS) algorithms, singular value decomposition (SVD) algorithms, squeeze-and-excitation (SE) network algorithms, principal component analysis (PCA) algorithms. The dimensionality reduction projects pattern of proximities among a set of features (e.g., geometric properties and/or chemical properties) by providing feature values and distances between the feature values.

In some embodiments, the surface patches having geometric properties and chemical properties (also referred to as input features to the neural network) can be normalized to be in a range from −1 to 1 to reduce errors and allow assignment user-defined feature weights used during neural network optimization.

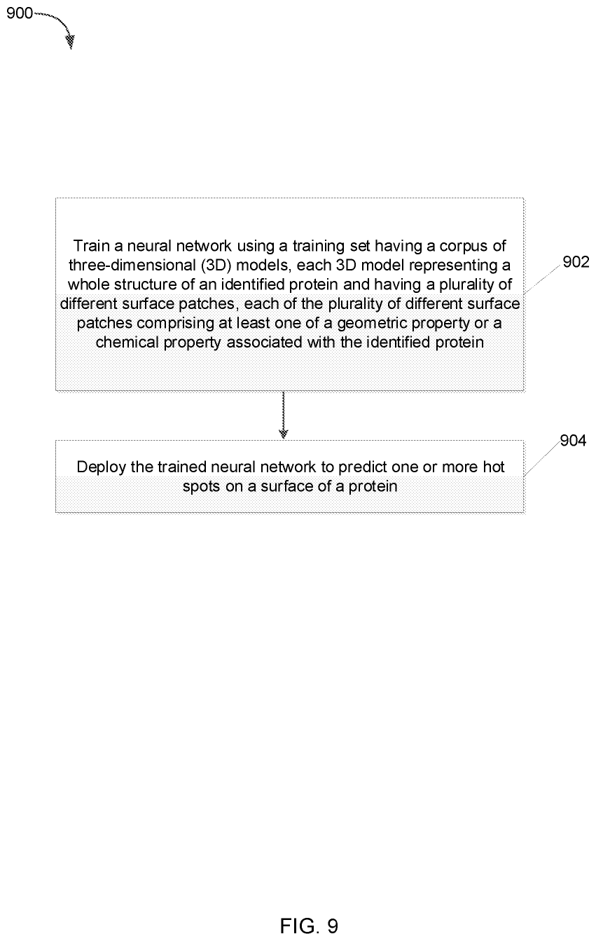

In some embodiments, instead of using arbitrary radii for determining the surface patches, the system 100 can use interaction patches. An interaction patch can be defined as a collection of surface points of a coherent region of a particular type (e.g., positive charge, negative charge, hydrophobicity) involved in protein-protein interactions. For example, in some embodiments, a hydrophobic patch can be calculated by projecting a hydrophobic potential of each atom onto a protein surface. A positive patch can be calculated by projecting a positive hydrophilic potential of each atom onto a protein surface. A negative patch can be calculated by projecting a negative hydrophilic potential of each atom onto a protein surface. An example of applying this concept in the context of identifying and predicting protein aggregation hot spots has been published by Sankar et al., “AggScore: Prediction of aggregation-prone regions in proteins based on the distribution of surface patches,” Proteins (2018), 86:1147-1156. In some embodiments, the feature space of the neural network can be fed with information of members of the same interaction patch at an interaction radius 5.0 Å instead of patches based on arbitrary radii, which can also refine the neural network training as described with respect to FIG. 9.

In some embodiments, the system 100 can utilize an energy decomposition method (e.g., an eigenvalue decomposition method) to decompose an interaction energy matrix associated with a protein (e.g., an interaction matrix that includes residue information accounting for van der Waals energy, electrostatic energy, hydrogen bonds energy, hydrophobic interaction, or some combination thereof) into eigenvalues to identify residues within the protein which contribute significantly to the stability of the protein and/or having strong couplings to interact with a biomolecule. For example, the components of the eigenvector associated with the lowest eigenvalue indicate which residues are likely to be responsible for the stability and for the rapid folding of the protein. An example of this concept is discussed in Tiana et al., “Understanding the determinants of stability and folding of small globular proteins from their energetics,” Protein Science (2004), 13:113-124, in which the identification of driver residues for protein stabilization and folding and further substantiated in the prediction of antibody/antigen interactions in Peri et al., “Surface energetics and protein-protein interactions: analysis and mechanistic implications,” Scientific Reports (2016), 6:24035.

In some embodiments, the system 100 can adjust the density of the molecular surface representation (e.g., a surface grid density) to increase the resolution of the surface grid. Examples are described with respect to FIGS. 3A and 3B.

In some embodiments, the system 100 can keep hydrogen atoms during an entire process including model creation process, training process, deployment process and/or various applications, while conventional methods remove hydrogen atoms in the interface (e.g., surface patches involved in the PPIs). Keeping hydrogen atoms ensures consistent treatment of a protein system during the entire process, which results in higher precision in the electrostatics and enables implicit assessment of clashes and feasibility of the binding configuration.

FIG. 2B is a flowchart illustrating processing steps 210 carried out by the system of the present disclosure to predict a hot spot. In step 212, the system 100 receives a structure of a protein input by a user, for example, a 3D model as taught herein. Examples are described with respect to the 3D model of FIG. 1 and step 202 of FIG. 2A.

In step 214, the system 100 performs structure preparation including chain assignments, if needed, to calculate a molecular surface of a protein. Examples are described with respect to step 204 of FIG. 2A. In some embodiments, the input protein structure is a result of querying one or more public databases such as Protein Data Bank. However, protein structures available from public databases often contains incomplete information that is needed for a surface property calculation. Protein structure preparation for use as input as taught herein can be performed using a software application (e.g., Schrodinger Protein Wizard) which is able to complete the protein structure information by including, for example, the addition of hydrogens and possibly missing sidechain atoms, assignment of partial charges and the and charge adjustments based on the respective system pH (default is pH 7.1). In some embodiments where the input protein structure is composed of more than two input chains, the system 100 can perform a chain assignment in which the system 100 determines which of the input chains are in contact with each other and which combination of these interacting chains are used for the subsequent surface calculations.

In step 216, the system 100 performs a surface calculation to determine a plurality of different surface patches. Examples are described with respect to step 204 of FIG. 2A.

In step 218, the system 100 performs a property calculation to calculate geometric properties and chemical properties 220 of the protein, including shape index, electrostatics, hydrophobicity, and hydrogen bonds. Examples are described with respect to step 206 of FIG. 2A.

In step 222, the system 100 normalizes values of the calculated properties, for example, into a range [0 1], [−1 0], [−1 1] or other suitable normalization range. Examples are described with respect to FIGS. 4-6.

In step 224, the system 100 assigns the normalized values to one or more of the plurality of different surface patches (e.g., each surface patch or some of the surface patches). Examples are described with respect to step 206 of FIG. 2A. In some embodiments, the system 100 can assign non-normalized values to one or more of the plurality of different surface patches.

In step 226, the system 100 performs a geodesic reduction to convert the surface patches into input vectors for the neural network model 126. Geodesic reduction is a method of dimensionality reduction, which can be done by projecting the surface features (hydrogen bond donor/acceptor, electrostatic charge propensity, hydrophobicity propensity, curvature) into a two-dimensional format that is better suited as input for the neuronal vectors for the neural network model 126.

In step 228, the system 100 feeds the input vectors into the neural network model 126.

In step 232, the system 100 predicts one or more hot spots on a surface of the protein. Examples are described with respect to step 208 of FIG. 2A.

FIG. 3A illustrates example surface patches 330A and 330B (also referred to as patches) in a molecular surface representation 340. FIG. 3B is a zoomed in illustration of the surface patch 330A in FIG. 3A. A portion of the surface patch 330A overlaps with a portion of the surface patch 330B. The surface patches 330A and 330B have geodesic radii 332A and 332B (shown in FIG. 3A), respectively. It should be understood that the shapes of the surface patches 330A and 330B are for illustration purpose and the shapes of the surface patches can be different based on the protein.

FIG. 4A illustrates an example molecular surface representation 400A representing positive charge values on the protein in FIG. 3A. Each vertex of the mesh surface of the molecular surface representation 400A is assigned a charge value normalized between 0 (indicative of no charges) and 1 (the greatest normalized positive charge values indicative of strong positive charge).

FIG. 4B illustrates an example molecular surface representation 400B representing negative charge values on the protein in FIG. 3A. Each vertex of the mesh surface of the molecular surface representation 400A is assigned a charge value normalized between 0 (indicative of no charges) and 1 (the greatest normalized negative charge values indicative of strong negative charge). Those skilled in the art will appreciate that the use of other scales is also possible to represent a normalized charge value. For example (not shown in FIGS. 4A and 4B), the charge values can be normalized between −1 (the greatest normalized negative charge values indicative of strong negative charge) and 1 (the greatest normalized positive charge values indicative of strong positive charge).

FIG. 5 illustrates an example molecular surface representation 500 representing hydrophobic regions on the protein in FIG. 3A. Each vertex of the mesh surface of the molecular surface representation 500 is assigned a hydrophobicity scalar value based on atomic s log P propensity values. The hydrophobicity scalar values are normalized to be between 0 (indicative of no hydrophilicity) and 1 (the greatest normalized hydrophobicity scalar values indicative of strong hydrophobicity). In some embodiments (not shown in FIG. 5), the hydrophobicity scalar values can be normalized between −1 (the greatest normalized hydrophilicity scalar values indicative of strong hydrophilicity) and 1 (the greatest normalized hydrophobicity scalar values indicative of strong hydrophobicity).

FIG. 6A illustrates an example molecular surface representation 600A representing hydrogen-bond acceptor regions on the protein in FIG. 3A. All possible donors and acceptors with a surface exposure are considered for hydrogen bond calculation. In some embodiments, less than all possible donors and acceptors with surface exposure can be considered for hydrogen bond calculation. The hydrogen bond geometry and energies are calculated based on Equation (1) and FIG. 7. Values normalized between 0 (indicative of no acceptors) and 1 (the greatest normalized hydrogen-bond energy values for acceptor indicative of acceptors having strong hydrogen bond) were assigned to each vertex of the mesh surface of the molecular surface representation 600A.

FIG. 6B illustrates an example molecular surface representation 600B representing hydrogen-bond donor regions on the protein in FIG. 3A. All possible donors and acceptors with a surface exposure are considered for hydrogen bond calculation. In some embodiments, less than all possible donors and acceptors with a surface exposure can be considered for hydrogen bond calculation. The hydrogen bond geometry and energies are calculated based on Equation (1) and FIG. 7. Values normalized between 0 (indicative of no donors) and 1 (the greatest normalized hydrogen-bond energy values for donors indicative of donors having strong hydrogen bond) were assigned to each vertex of the mesh surface of the molecular surface representation 600B. In some embodiments (not shown in FIGS. 6A and 6B), negative values represent donors and positive values represent acceptors. For example, values can be normalized between −1 (the greatest normalized hydrogen-bond energy values for donors indicative of donors having strong hydrogen bond) and 1 (the greatest normalized hydrogen-bond energy values for acceptors indicative of acceptors having strong hydrogen bond).

FIG. 7 illustrates a hydrogen bond geometry 700 used in a hydrogen-bond energy potential given in Equation (1) as described above. θ is a donor (N: nitrogen atom) 704—hydrogen 706—acceptor (O: oxygen atom) 708 angle, ϕ is the hydrogen 706—acceptor 708—base (C: carbon atom) 710 angle, d is a donor 704—acceptor 708 distance, and r is a hydrogen 706—acceptor 708 distance. Γ (not shown in FIG. 7) is an angle between the normals to the planes defined by the bonds from the donor 704 and the acceptor 708.

FIG. 8 illustrates an example molecular surface representation 800 representing a protein interface surface on a protein. Each vertex of the mesh surface of the molecular surface representation 800 is assigned a value indicative of a corresponding atom in contact with other chains of the protein. Only surface vertices of atoms that are in contact with other chains are considered for an interface prediction that is a prediction of surfaces patches involved in PPIs.

FIG. 9 is an example flowchart illustrating neural network training steps 900 carried out by the system 100 of the present disclosure.

In step 902, the system 100 trains a neural network using a training set having a corpus of 3D models. Each 3D model represents a whole structure of an identified protein (e.g., the protein structure described above with respect to FIG. 1) in the context of PPI. Each 3D model has a plurality of different surface patches. Each of the plurality of different surface patches includes at least one of a geometric property or a chemical property associated with the identified protein. For example, the neural network training engine 120 of the system 100 can carry out the training steps 900. The training set generator 122 can obtain whole protein structures from an external source (e.g., a public source/database). In some embodiments, the whole protein structures can be based on a protein benchmark used in protein/protein docking (such as protein benchmark V5) (e.g., Vreven, T. et al., Journal of Molecular Biology, 2015, vol. 427, p. 3031-41)

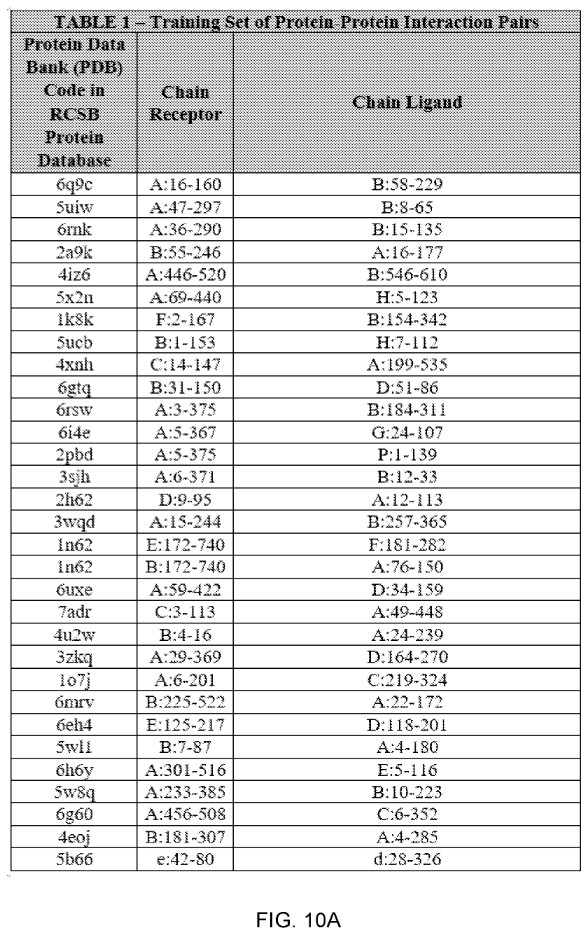

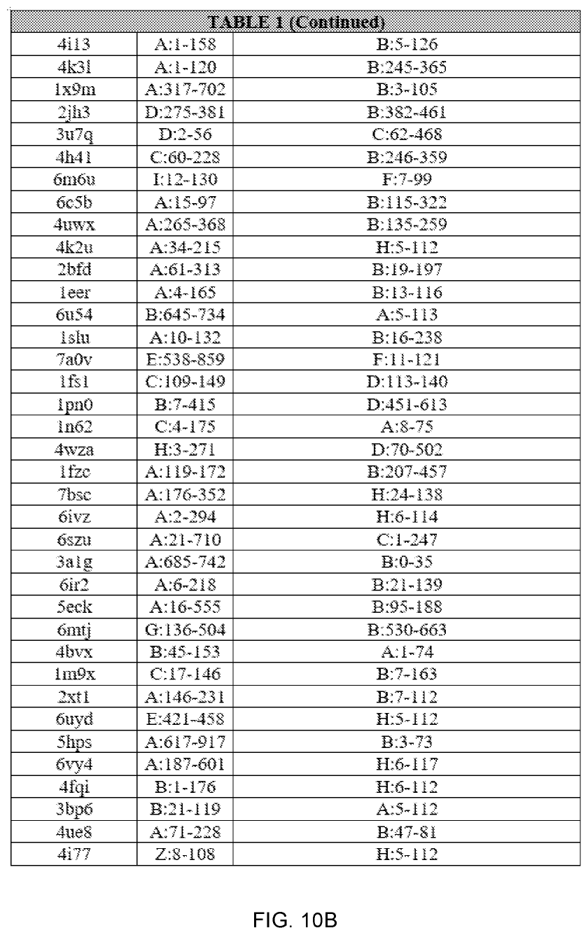

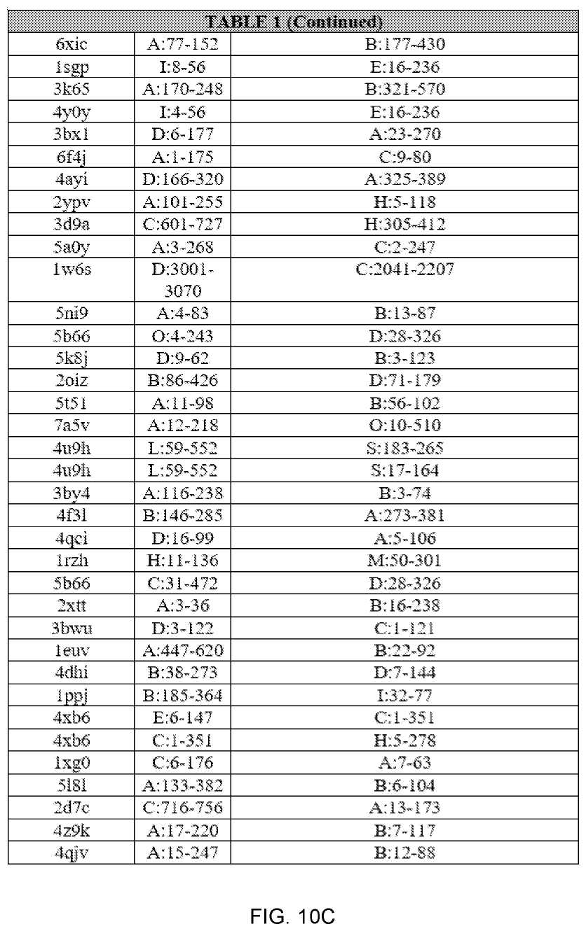

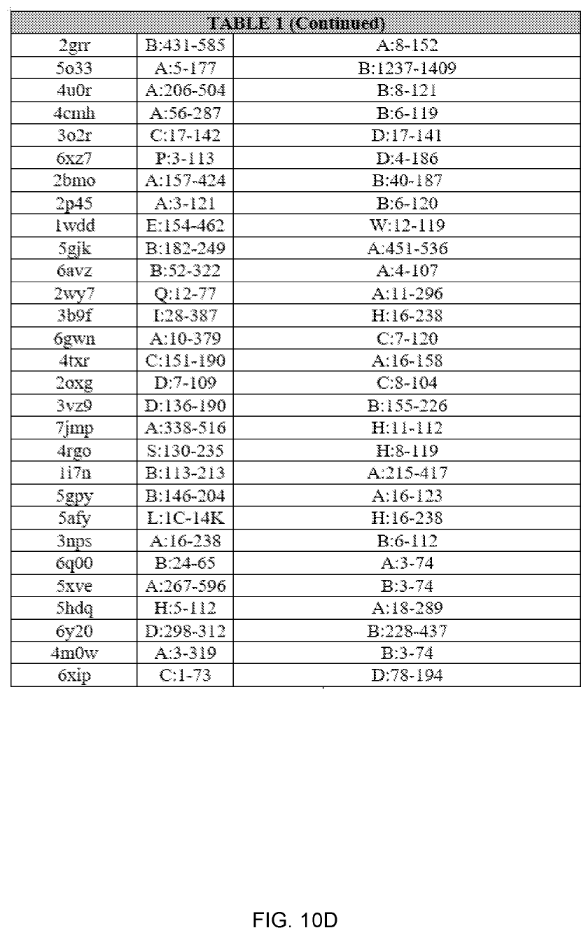

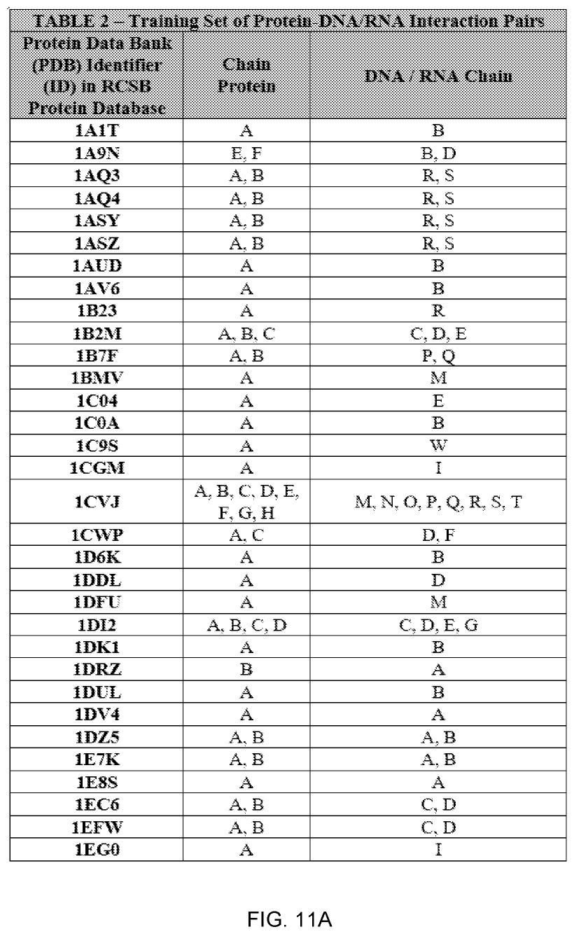

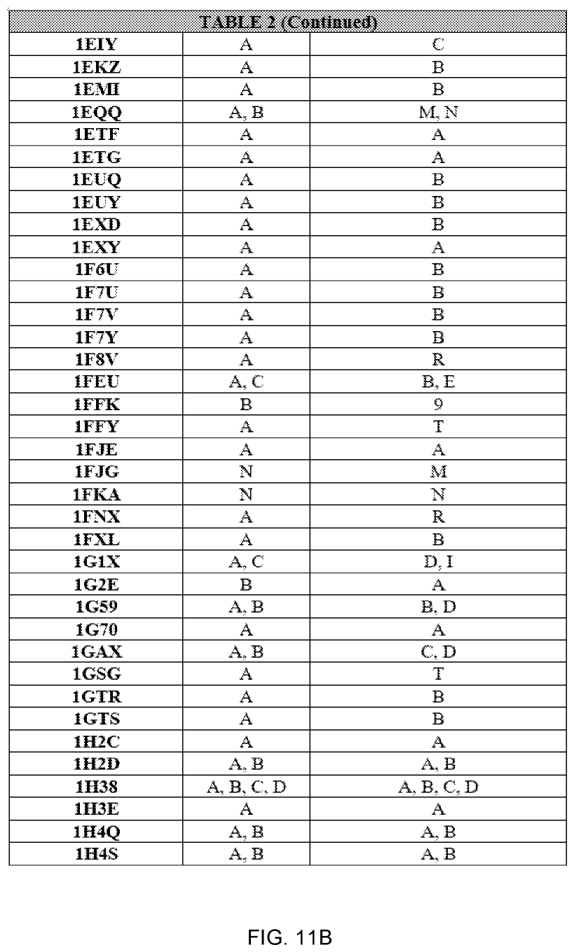

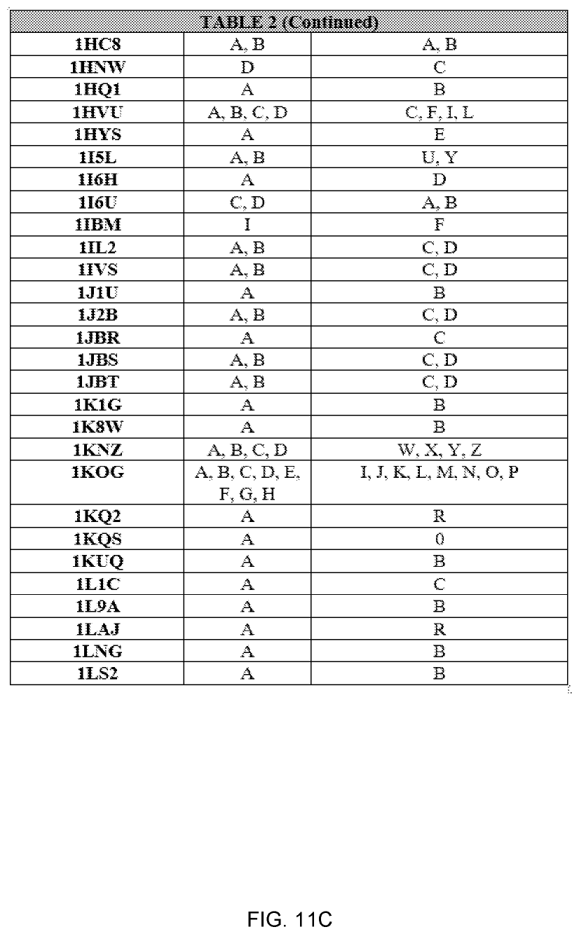

The training set generator 122 can generate training sets including, but not limited to, the 3D models of various whole protein structures, surface patches with known/calculated geometric properties and/or chemical properties for a particular 3D model, calculated geometric properties and/or chemical properties for a particular surface patch, molecular surfaces with known/calculated geometric properties and/or chemical properties for a particular 3D model, known/labeled/identified interacting protein pairs having a binder protein and a target protein for various protein interaction scenarios (e.g., weak PPIs, strong PPIs, and other suitable PPIs), other known/labeled/identified interacting protein-biomolecule pairs for various protein-biomolecule interaction scenarios including but not limited to protein-protein interaction pairs listed in Table 1 shown in FIGS. 10A-10D, other protein-biomolecule interaction pairs (e.g., protein-deoxyribonucleic acid (DNA)/ribonucleic acid (RNA) interaction pairs listed in Table 2 shown in FIGS. 11A-11C, known/labeled/identified interacting patch pairs having binder patches and target patches, known/labeled/identified noninteracting set having target proteins/biomolecules/patches and random proteins/biomoleculers/patches, a known/labeled/identified interaction type associated with the above data, and other suitable application-specific training data. In some embodiments, the known/labeled/identified interacting protein-biomolecule pairs, known/labeled/identified interacting patch pairs and/or known/labeled/identified noninteracting set may be found from a public source/database like the RCSB Protein Database. In some embodiments, ligands, DNAs, metals, and/or crystal contracts can be removed from the training sets.

The training module 124 can feed the training sets into a neural network to be trained. For example, the training module 124 can feed interacting proteins (e.g., two single-chain proteins having no ligand, no DNA, no metal, and/or no crystal contacts), other interacting protein-biomolecule pairs, noninteracting proteins, and/or other noninteracting protein-biomolecule groups into the neural network. The training module 124 can adjust the weights and other parameters in the neural network during the training process to reduce the difference between an output of the neural network and an expected output. The trained neural networks can be stored in the database 104 or the neural network module 126.

In step 904, the system 100 deploys the trained neural network to predict one or more hot spots on a surface of a protein. For example, the neural network training engine 120 can select a group of training sets as validation sets and apply the trained neural network to the validation sets to evaluate the trained neural network. In an another example, the system 100 can deploy the trained neural network to predict hot spots on a surface of a protein (e.g., an unidentified protein, a protein input by a user, an unknown protein or a random protein).

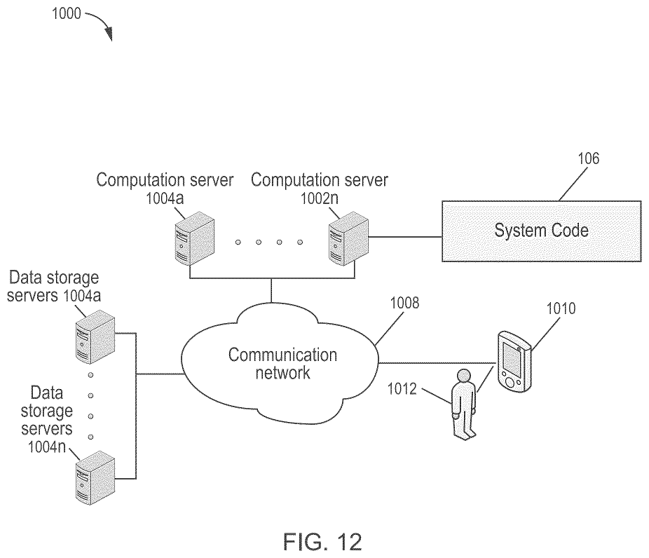

FIG. 12 is an example diagram illustrating computer hardware and network components on which the system 1000 can be implemented. The system 1000 can include a plurality of computation servers 1002a-1002n having at least one processor (e.g., one or more graphics processing units (GPUs), microprocessors, central processing units (CPUs), tensor processing units (TPUs), application-specific integrated circuits (ASICs), etc.) and memory for executing the computer instructions and methods described above (which can be embodied as system code 106). The system 1000 can also include a plurality of data storage servers 1004a-1004n for storing data. The computation servers 1002a-1002n, the data storage servers 1004a-1004n, and the user device 1010 can communicate over a communication network 1008. Of course, the system 1000 needs not be implemented on multiple devices, and indeed, the system 1000 can be implemented on a single device (e.g., a personal computer, server, mobile computer, smart phone, etc.) without departing from the spirit or scope of the present disclosure.

FIG. 13 is an example block diagram of an example computing device 102 that can be used to perform one or more steps of the methods provided by example embodiments. The computing device 102 includes one or more non-transitory computer-readable media for storing one or more computer-executable instructions or software for implementing example embodiments. The non-transitory computer-readable media can include, but are not limited to, one or more types of hardware memory, non-transitory tangible media (for example, one or more magnetic storage disks, one or more optical disks, one or more USB flashdrives), and the like. For example, memory 1106 included in the computing device 102 can store computer-readable and computer-executable instructions or software for implementing example embodiments. The computing device 102 also includes processor 1102 and associated core 1104, and optionally, one or more additional processor(s) 1102′ and associated core(s) 1104′ (for example, in the case of computer systems having multiple processors/cores), for executing computer-readable and computer-executable instructions or software stored in the memory 1106 and other programs for controlling system hardware. Processor 1102 and processor(s) 1102′ can each be a single core processor or multiple core (1104 and 1104′) processor. The computing device 102 also includes a graphics processing unit (GPU) 1105. In some embodiments, the computing system 102 includes multiple GPUs.

Virtualization can be employed in the computing device 102 so that infrastructure and resources in the computing device can be shared dynamically. A virtual machine 1114 can be provided to handle a process running on multiple processors so that the process appears to be using only one computing resource rather than multiple computing resources. Multiple virtual machines can also be used with one processor.

Memory 1106 can include a computer system memory or random access memory, such as DRAM, SRAM, EDO RAM, and the like. Memory 1106 can include other types of memory as well, or combinations thereof. A user can interact with the computing device 102 through a visual display device 1118, such as a touch screen display or computer monitor, which can display one or more user interfaces 1119. The visual display device 1118 can also display other aspects, transducers and/or information or data associated with example embodiments. The computing device 102 can include other I/O devices for receiving input from a user, for example, a keyboard or any suitable multi-point touch interface 1108, a pointing device 1110 (e.g., a pen, stylus, mouse, or trackpad). The keyboard 1108 and the pointing device 1110 can be coupled to the visual display device 1118. The computing device 102 can include other suitable conventional I/O peripherals.

The computing device 102 can also include one or more storage devices 1124, such as a hard-drive, CD-ROM, or other computer readable media, for storing data and computer-readable instructions, applications, and/or software that implements example operations/steps of the system (e.g., the systems 100 and 1000) as described herein, or portions thereof, which can be executed to generate user interface 1119 on display 1118. Example storage device 1124 can also store one or more databases for storing any suitable information required to implement example embodiments. The databases can be updated by a user or automatically at any suitable time to add, delete or update one or more items in the databases. Example storage device 1124 can store one or more databases 1126 for storing provisioned data, and other data/information used to implement example embodiments of the systems and methods described herein.

The system code 106 as taught herein may be embodied as an executable program and stored in the storage 1124 and the memory 1106. The executable program can be executed by the processor to perform the in-situ inspection as taught herein.

The computing device 102 can include a network interface 1112 configured to interface via one or more network devices 1122 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 1112 can include a built-in network adapter, network interface card, PCMCIA network card, card bus network adapter, wireless network adapter, USB network adapter, modem or any other device suitable for interfacing the computing device 102 to any type of network capable of communication and performing the operations described herein. Moreover, the computing device 102 can be any computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

The computing device 102 can run any operating system 1116, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In some embodiments, the operating system 1116 can be run in native mode or emulated mode. In some embodiments, the operating system 1116 can be run on one or more cloud machine instances.

It should be understood that the operations and processes described above and illustrated in the figures can be carried out or performed in any suitable order as desired in various implementations. Additionally, in certain implementations, at least a portion of the operations can be carried out in parallel. Furthermore, in certain implementations, less than or more than the operations described can be performed.

In describing example embodiments, specific terminology is used for the sake of clarity. For purposes of description, each specific term is intended to at least include all technical and functional equivalents that operate in a similar manner to accomplish a similar purpose. Additionally, in some instances where a particular example embodiment includes multiple system elements, device components or method steps, those elements, components or steps may be replaced with a single element, component or step. Likewise, a single element, component or step may be replaced with multiple elements, components or steps that serve the same purpose. Moreover, while example embodiments have been shown and described with references to particular embodiments thereof, those of ordinary skill in the art will understand that various substitutions and alterations in form and detail may be made therein without departing from the scope of the present disclosure. Further still, other embodiments, functions and advantages are also within the scope of the present disclosure.

What is claimed is:

1. A computer-implemented method for predicting one or more hot spots on a surface of a protein, the method comprising:
receiving a three-dimensional (3D) model representing a whole structure of a protein;
determining a plurality of different surface patches associated with the 3D model;
determining for at least one of the plurality of different surface patches at least one of a geometric property or a chemical property;
assigning each node of a first surface patch of the plurality of surface patches a plurality of input features, the plurality of input features comprising one or more chemical features; and
processing, with a neural network, at least one of a collection of geometric properties collected from one or more of the plurality of different surface patches or a collection of the chemical properties collected from one or more of the plurality of different the surface patches to predict one or more hot spots on the surface of the protein.

2. The computer-implemented method of claim 1, wherein the geometric property comprises a shape index and a distance-dependent curvature.

3. The computer-implemented method of claim 1, wherein the chemical property comprises an atom partial charge assigned by a protein force field, an atom radius assigned by the protein force field, an atomic s log P propensity value independent of natural amino acid context, a negative value representing a donors, a positive value representing an acceptor, a hydrogen bond geometry defined by an established force field definition, a hydrogen bond energy defined by the established force field definition.

4. The computer-implemented method of claim 1, wherein each of the plurality of different surface patches comprises a collection of surface points with similar chemical properties.

5. A computer-implemented method for training a neural network for predicting one or more hot spots on a surface of a protein, comprising:
training a neural network using a training set having a corpus of three-dimensional (3D) models, each 3D model representing a whole structure of an identified protein and having a plurality of different surface patches, each of the plurality of different surface patches comprising at least one of a geometric property or a chemical property associated with the identified protein; and
deploying the trained neural network to predict the one or more hot spots on the surface of the protein.

6. A system for predicting one or more hot spots on a surface of a protein, the system comprising:
a memory storing one or more instructions;
a processor configured to or programmed to execute the one or more instructions stored in the memory in order to:
receive a three-dimensional (3D) model representing a whole structure of a protein;
determine a plurality of different surface patches associated with the 3D model;
determine for at least one of the plurality of different surface patches at least one of a geometric property or a chemical property;
assign each node of a first surface patch of the plurality of surface patches a plurality of input features, the plurality of input features comprising one or more chemical features; and
process, with a neural network, at least one of a collection of geometric properties collected from one or more of the plurality of different surface patches or a collection of the chemical properties collected from one or more of the plurality of different the surface patches to predict one or more hot spots on the surface of the protein.

7. The system of claim 6, wherein the geometric property comprises a shape index and a distance-dependent curvature.

8. The system of claim 6, wherein the chemical property includes at least one of: an atom partial charge assigned by a protein force field, an atom radius assigned by the protein force field, an atomic s log P propensity value independent of natural amino acid context, a negative value representing a donors, a positive value representing an acceptor, a hydrogen bond geometry defined by an established force field definition, or a hydrogen bond energy defined by the established force field definition.

9. The system of claim 6, wherein each of the plurality of different surface patches comprises a collection of surface points with similar chemical properties.

10. The system of claim 6, wherein the processor is configured to execute instructions to:
train a neural network using a training set having a corpus of three-dimensional (3D) models, each 3D model representing a whole structure of an identified protein and having a plurality of different surface patches, each of the plurality of different surface patches comprising at least one of a geometric property or a chemical property associated with the identified protein; and
deploy the trained neural network to predict the one or more hot spots on the surface of the protein.
